(19) United States
(12) Patent Application Publication
(10) Pub. No.: US 2018/0085212 A1
(43) Pub. Date: Mar. 29, 2018

(54) OCULAR FUNCTION ASSISTANCE DEVICE

(71) Applicant: KABUSHIKI KAISHA TOPCON, Itabashi-ku (JP)

(72) Inventors: Toshifumi MIHASHI, Yokohama-shi (JP); Yasufumi FUKUMA, Wako-shi (JP); Makoto FUJINO, Itabashi-ku (JP)

(73) Assignee: KABUSHIKI KAISHA TOPCON, Itabashi-ku (JP)

(21) Appl. No.: 15/554,313

(22) PCT Filed: Feb. 22, 2016
(86) PCT No.: PCT/JP2016/055023

§ 371 Date: Aug. 29, 2017

(30) Foreign Application Priority Data
Mar. 13, 2015 (JP) 2015-050345

(51) Int. Cl. A61F 2/16

(52) U.S. Cl. CPC A61F 2/1624

(57) ABSTRACT

An ocular function assistance device of an embodiment includes an actuator, a controller, and an information input unit. The ocular function assistance device is used for assisting an ocular function. The actuator receives electricity and operates for providing a predefined ocular function. The controller executes at least control of electricity supply to the actuator. The information input unit inputs biological information or condition information into the controller. The controller changes a control mode of the actuator based on the biological information or the condition information.

### TECHNICAL FIELD

Embodiments described herein relate generally to an ocular function assistance device.

### BACKGROUND ART

The sense of vision is thought to give the greatest influence on the quality of life (QOL) among various human senses, and the disorder of the sense of vision enormously deteriorates QOL. Hence, it is important to establish techniques of complementing the lost ocular function. Intraocular lenses (IOL) are known as such techniques.

The patent document 1 discloses a technique of correcting the astigmatism or one or more higher order aberrations by implanting an intraocular lens into the capsular bag of the eye and by changing the tension of the surface of the intraocular lens and the shape of the intraocular lens.

In addition, the patent document 2 discloses an intraocular lens including an outer shell configured to promote bonding with the capsular bag, and a force transfer assembly configured to transfer forces from the capsular bag to change the shape of the outer shell filled with a fill material in response to changes in the shape of the capsular bag.

### PRIOR ART DOCUMENTS

### Patent Documents

### [Patent Document 1] Japanese Patent No. 5027119

### [Patent Document 2] Japanese Unexamined Patent Application Publication (Translation of PCT Application) No. 2014-521394

### SUMMARY OF THE INVENTION

### Problems to be Solved by the Invention

However, such conventional techniques have a problem that the ocular function is difficult to be properly maintained when various circumstances changes in daily life.

In order to solve such a problem, the purpose of the present invention is to provide a novel technique to assist the ocular function.

### Means of Solving the Problems

An ocular function assistance device of an embodiment is used for assisting an ocular function. The ocular function assistance device includes an actuator, a controller, and an information input unit. The actuator receives electricity and operates for providing a predefined ocular function. The controller executes at least control of electricity supply to the actuator. The information input unit inputs biological information or condition information into the controller. The controller changes a control mode of the actuator based on the biological information or the condition information.

### Effects of the Invention

According to the present invention, a novel technique to assist the ocular function can be provided.

### BRIEF DESCRIPTION OF THE DRAWINGS

FIG. 1 is a schematic diagram showing an example of the configuration of the ocular function assistance device of the embodiment.

FIG. 2 is a schematic diagram showing an example of the configuration of the ocular function assistance device of the embodiment.

FIG. 3 is a schematic diagram showing an example of the configuration of the ocular function assistance device of the embodiment.

FIG. 4A is a schematic diagram showing an example of the configuration of the ocular function assistance device of the embodiment.

FIG. 4B is a schematic diagram showing an example of the configuration of the ocular function assistance device of the embodiment.

FIG. 5 is a schematic diagram showing an example of the configuration of the ocular function assistance device of the embodiment.

FIG. 6 is a flow chart showing an example of the operation of the ocular function assistance device of the embodiment.

FIG. 7 is a flow chart showing an example of the operation of the ocular function assistance device of the embodiment.

FIG. 8 is a flow chart showing an example of the operation of the ocular function assistance device of the embodiment.

FIG. 9 a schematic diagram showing an example of the configuration of the ocular function assistance device of the embodiment.

FIG. 10 a schematic diagram showing an example of the configuration of the ocular function assistance device of the embodiment.

### DESCRIPTION OF THE EMBODIMENTS

Exemplary embodiments of ocular function assistance devices according to the present invention will be described in detail with referring to the drawings. The ocular function assistance devices according to the embodiments are used to assist ocular function devices or intraocular sites providing a predetermined ocular function. Examples of such ocular function devices include devices arranged inside eyes such as intraocular lenses, artificial irises, and artificial retinas, and devices put on eyes such as adjustable glasses and contact lenses. Examples of such intraocular sites to be assisted include crystalline lenses, irises, and retinas. Note that any of the contents described in the documents cited in the present specification may be applied to the embodiments below.

Hereinafter, cases in which ocular function assistance devices of embodiments assist the ocular refraction function using intraocular lenses will be described

### <Ocular Function Assistance Device>

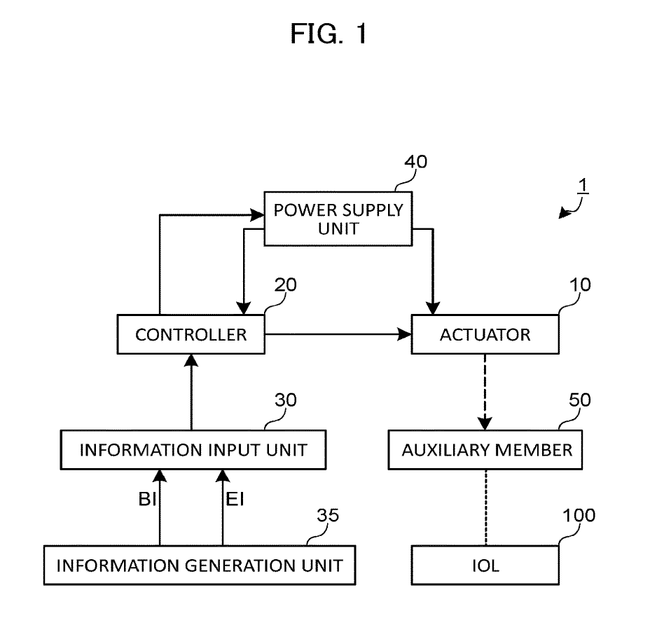

FIG. 1 shows a block diagram of an example of the configuration of the ocular function assistance device according to the embodiment. The ocular function assistance device 1 assists the ocular refraction function provided by the intraocular lens placed in the capsular bag of the eye. The ocular function assistance device 1 assists the ocular refraction function provided by the intraocular lens to provide the ocular refractive power regulation function.

The ocular function assistance device 1 includes the actuator 10 and the controller 20. The ocular function assistance device 1 may further include at least one of the information input unit 30 and the power supply unit 40.

### <Actuator>

The actuator 10 receives electricity and operate for providing the ocular refractive power regulation function. The actuator 10 is configured to be capable of bending in the direction corresponding to the electric current direction (polarity) upon receiving power supply. The actuator 10 can adjust the degree of deformation in accordance with the electricity supplied. Note that the actuator 10 may be configured to adjust the degree of deformation according to electric currents and/or voltages. In the present embodiment, the auxiliary member 50 and the intraocular lens 100 are placed in the optical path of the light incident on the eye through the pupil. The deformation of the actuator 10 acts on the auxiliary member 50, and the deformation of the actuator 10 changes the shape of the auxiliary member 50. The deformation of the actuator 10 changes the shape of the auxiliary member 50, which is placed in the optical path of the light incident on the eye through the pupil, thereby changing the ocular refractive power.

For example, the actuator 10 may be configured to include a polymeric material (e.g., flexible polymer, stretchable polymer, elastic polymer) whose shape changes upon receiving electricity and a pair of electrodes that interposes the polymeric material in between, and to be capable of deforming according to the voltage applied to the pair of electrodes. The actuator 10 thus configured may be the ionic polymer actuator disclosed in Japanese Patent No. 5594690 or the carbon nanofiber actuator disclosed in Japanese Unexamined Patent Application Publication No. 2013-34368, for example.

### <Controller>

The controller 20 receives electricity from the power supply unit 40 and controls at least part of the ocular function assistance device 1. For instance, the controller 20 executes the control of the actuator 10. Examples of the actuator 10 include the switching of the operation ON and the operation OFF, and the control of the value of the electricity supplied to the actuator 10. In addition, the controller 20 can control the actuator 10 or the power supply unit 40 to control the power supply to the actuator 10 and the direction of the electric current supplied to the actuator 10. The controller 20 includes, for example, arbitrary kinds of electric circuits that include arbitrary number of electric components such as a resistor, a transistor, a capacitor, and an inductor.

### <Information Input Unit>

The information input unit 30 receives biological information BI or condition information EI, and inputs the biological information BI or the condition information EI into the controller 20. The information input unit 30 may be configured to input both the biological information BI and the condition information EI. The biological information BI includes, for example, the convergence state of eye balls, the direction of line of sight, the brain wave, the pupil diameter, electromyogram, the movement of a muscle, or the like. The condition information EI includes, for example, the ambient lightness (brightness). The biological information BI or the condition information EI is generated, for example, by the information generation unit 35 arranged outside the ocular function assistance device 1.

The information generation unit 35 includes, for example, a sensor, an imaging device, or the like. The sensor detects the size of the ciliary body, the muscle potential of the ciliary body, the action potential of the short ciliary nerve, the brain wave, or the like. The imaging device acquires an image for detecting the direction of line of sight, the pupil diameter, the pupil distance, the distance to the target (object) to be watched by the eye, or the like. Based on the detection signal obtained by the sensor, the image acquired by the imaging device, or the like, the information generation unit 35 generates the biological information BI or the condition information EI. The information input unit 30 may include functions of at least part of the information generation unit 35. The information input unit 30 may include a signal reception unit that receives a wired or wireless signal corresponding to the biological information BI or the condition information EI from the outside. The information input unit 30 may include a light reception unit that receives light including information corresponding to the biological information BI or the condition information EI.

### <Power Supply Unit>

The power supply unit 40 supplies electricity to each part of the ocular function assistance device 1. For example, the power supply unit 40 supplies electricity to the controller 20 and the actuator 10. The power supply unit 40 supplies, for example, a voltage of 5 V and an electric current of 1 mA or less. The power supply unit 40 may use a solar cell, the electromagnetic induction, a lithium ion battery, an intracerebral current, or a sending current. The power supply unit 40 may include an electricity supply controller that executes the control of electricity supply. A configuration may be employed in which the power supply unit 40 includes a secondary battery and an external terminal, and the secondary battery can be charged via the external terminal at an arbitrary timing.

### <Auxiliary Member>

The auxiliary member 50 is placed in the eye and provides the ocular refractive power regulation function upon receiving the operation of the actuator 10. The auxiliary member 50 has flexibility. The auxiliary member 50 transmits at least light entering the eye among light entering the eye. The curvature of the surface of the auxiliary member 50 can be changed by the change in the shape of the auxiliary member 50 according to the change in the shape of the actuator 10. The auxiliary member 50 thus configured may be a member in which a transparent material with viscoelasticity is enclosed in a transparent and deformable envelope, or the like. The material enclosed in the envelope may be silicon, water, or air.

### <Intraocular Lens>

The intraocular lens 100 is placed in the eye and includes a lens having refractive power of about 20 diopters. The intraocular lens 100 may include a liquid crystal lens, an Alvarez lens, a fluid lens, an artificial crystalline lens implanted in the eye instead of the removed crystalline lens, a lens implanted in the eye with the crystalline lens remained, or the like. For example, the intraocular lens 100 has a transparent area that transmits at least light of visible wavelength bands. The diameter of the transparent area is, for example, 2 mm, and the maximum diameter thereof is 8 mm.

As described above, the auxiliary member 50 and the intraocular lens 100 are placed in the optical path of the light entering the eye through the pupil. The controller 20 receives electricity from the power supply unit 40, and controls the actuator 10 to bend the actuator 10. When the actuator 10 is deformed, the auxiliary member 50 is also deformed, thereby changing the curvature of the surface of the auxiliary member 50 through which the light entering the eye passes. As a result, the regulation of the ocular refractive power can be performed.

The auxiliary member 50 and the intraocular lens 100 may be integrally formed. The intraocular lens 100 may have flexibility like the auxiliary member 50, and the auxiliary member 50 and the intraocular lens 100 may be foldable. With this, the implantation into the eye becomes easier.

The controller 20 can change the operation mode of the actuator 10 based on the biological information BI or the condition information EI input by the information input unit 30. For example, regardless of the content of the type of the biological information BI, the operation mode of the actuator 10 is changed according to the type of the condition information EI. Specifically, based on the condition information EI, the controller 20 determines the ambient brightness that is a factor of changing the pupil diameter. If the brightness is equal to or larger than a threshold (that is, if the pupil diameter is assumed to be small), the actuator 10 is controlled so as not to change the ocular refractive power. Conversely, the operation mode of the actuator 10 can be changed according to the type of the biological information BI, regardless of the content of the condition information EI.

The controller 20 can switch the operation mode between an ordinary mode and an electricity supply stop mode. For example, the controller 20 can stop the electricity supply to the actuator 10 when the information input unit 30 inputs predefined first information as the biological information BI or the condition information EI. The first information may be set in an arbitrary manner. The first information may be set based on past control contents (e.g., control history, control record). With this, it becomes possible to avoid unnecessary electricity supply to the actuator 10, and to significantly reduce electricity consumption for continuing to maintain the ocular refractive power regulation function.

In addition, the controller 20 executes control in such a manner that the actuator 10 changes the refractive power of the intraocular lens 100 (or the crystalline lens) from a reference value corresponding to the ocular refractive power of minus 1 diopter. For example, by setting the state in which the ocular refractive power is minus 1 diopter to be the resting state of accommodation, it becomes unnecessary to operate the actuator 10. As a result, it becomes unnecessary for the controller 20 to control the actuator 10, and therefore, further reduction in electricity consumption can be achieved. Note that the controller 20 may be configured to store, in a storage device (not illustrated), the control content that has been applied when the electricity supply to the actuator 10 has been stopped in the resting state of accommodation, as the reference value. According to this, it becomes possible to learn the electricity supply stop states for individual subjects and perform electricity saving according to individual subjects.

Further, the controller 20 may be configured to switch the operation mode between a coarse operation mode for coarsely operating the actuator 10 and a fine operation mode for finely operating the actuator 10, based on the biological information BI or the condition information EI input by the information input unit 30. For example, if the condition information EI shows that the brightness is smaller than a threshold (that is, the pupil diameter is assumed to be large), the actuator 10 is controlled to perform the fine operation of the ocular refractive power. Conversely, if the condition information EI shows that the brightness is equal to or larger than the threshold (that is, the pupil diameter is assumed to be small), the actuator 10 is controlled to perform the coarse operation of the ocular refractive power. As a result, it becomes possible to appropriately assist the ocular refractive power regulation function according to the change of the environmental conditions.

Note that at least one of the controller 20, the information input unit 30, and the power supply unit 40 may be arranged outside the eye.

Specific examples of the configuration of the ocular function assistance device 1 of the present embodiment will be described below. Hereinafter, as for the surface of the auxiliary member 50, the surface on the cornea side is referred to as the front face, and that on the retina side is referred to as the rear face.

### Configuration Examples

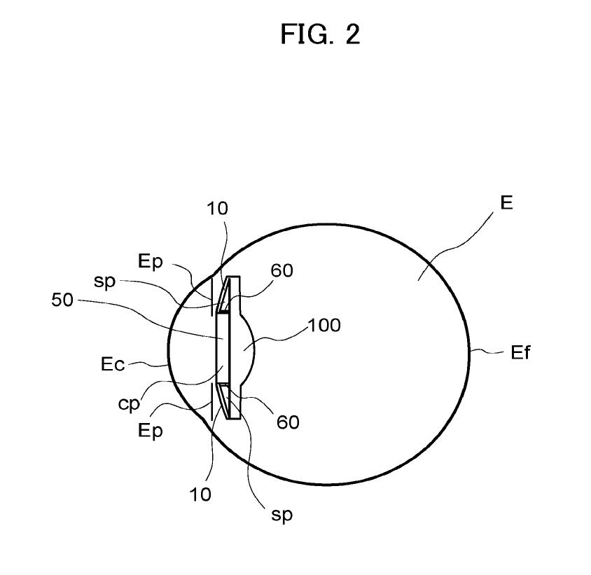

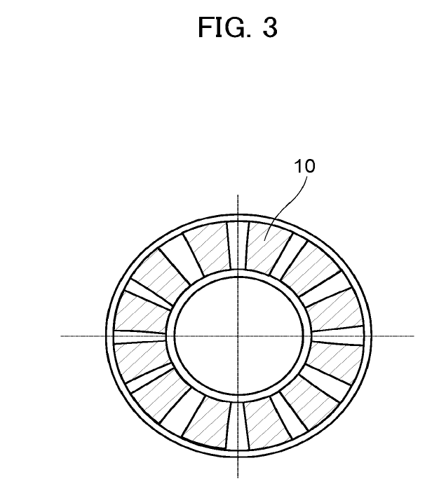

FIG. 2 shows a schematic sectional view of the eye in which the ocular function assistance device 1 of the present embodiment is implanted. FIG. 3 shows a schematic diagram of an arrangement example of the actuator 10 of the present embodiment. FIG. 3 is a view of the actuator 10 of the present embodiment as seen from the front side of the eye. The symbol Ec shows the cornea of the eye E, the symbol Ep shows the iris of the eye E, and the symbol Ef shows the retina of the eye E. Note that FIG. 2 mainly illustrates the actuator 10 and the auxiliary member 50, and the same parts as in FIG. 1 are shown by the same symbols and their descriptions will be omitted in an appropriate manner.

The optical path of the light entering the eye is provided with the auxiliary member 50 and the intraocular lens 100 in the order from the cornea side to the retina side. The auxiliary member 50 is arranged such that the auxiliary member 50 contacts the lens part the intraocular lens 100. The light that has passed through the pupil further passes through the auxiliary member 50 and the lens part the intraocular lens 100, and then is projected on the retina.

As shown in FIG. 3, one or more actuators 10 are placed in the peripheral part of the auxiliary member 50. The actuator 10 is arranged on the surface (front face) of the auxiliary member 50. For example, one or more support parts 60 stand on the intraocular lens 100. One or more apertures are formed in the support part 60. With this, the space cp in the auxiliary member 50 in the region where the actuator 10 is arranged on the surface is in communication with the space cp in the auxiliary member 50 in the passing region through which the light entering the eye passes. One end of the actuator 10 is held by the edge of the auxiliary member 50, and the other end is supported by the support part 60.

As an example, the refractive index of cornea is 1.376, the refractive index of hydatoid is 1.3374, the refractive index of the substance in the auxiliary member 50 is 1.428 (Adrian's study), the refractive index of the intraocular lens 100 is 1.47, and the refractive index of vitreous body is 1.336.

Note that the other end of the actuator 10 is supported by the support part 60 in FIG. 2; however, a configuration may be employed in which only one end of the actuator 10 is fixed on the edge part of the auxiliary member 50.

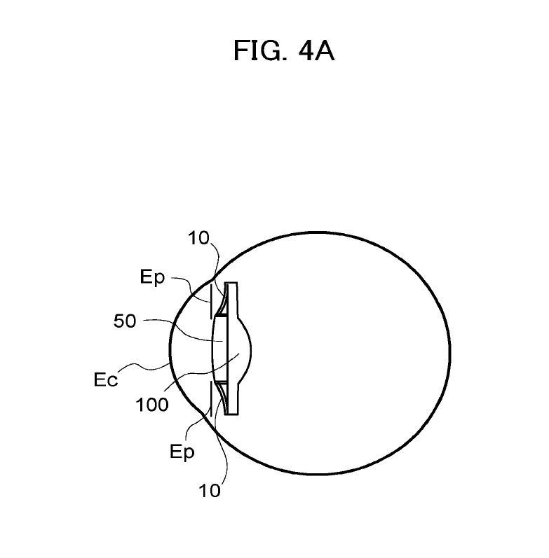

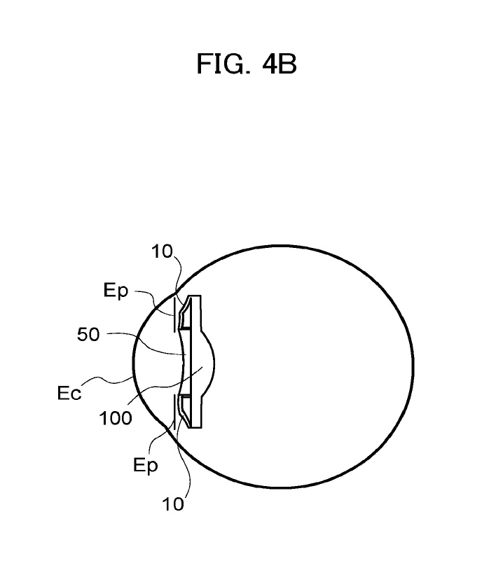

FIGS. 4A and 4B show operation description diagrams of the ocular function assistance device 1. As with FIG. 2, FIGS. 4A and 4B show schematic sectional views of the eye. FIG. 4A shows an operation description diagram when the auxiliary member 50 becomes thick in the passing direction of the light entering the eye. FIG. 4B shows an operation description diagram when the auxiliary member 50 becomes thin in the passing direction of the light entering the eye. Note that in FIGS. 4A and 4B, the same parts as in FIG. 2 are shown by the same symbols and their descriptions will be omitted in an appropriate manner.

The controller 20 (not illustrated in FIGS. 4A and 4B) controls the actuator 10 to bend the actuator arranged on the peripheral part of the auxiliary member 50. For example, when the actuator 10 is bent so as to push out the front face of the auxiliary member 50 as shown in FIG. 4A, the deformation of the front face of the auxiliary member 50 toward the cornea side is restrained by the intraocular lens 100, and the deformation in the above passing region in the direction opposite to the intraocular side. In other words, the bending of the auxiliary member 50 increases the thickness of the central part (passing region) of the auxiliary member 50 in the light passing direction, and the curvature of the front face of the auxiliary member 50 changes.

In addition, for example, when the actuator 10 is bent so as to pull in the front face of the auxiliary member 50 as shown in FIG. 4B, the front face in the above passing region become hollow toward the retina side. In other words, the bending of the auxiliary member 50 decreases the thickness of the central part of the auxiliary member 50 in the light passing direction, and the curvature of the front face of the auxiliary member 50 changes.

As described above, the curvature of the passing region of the light entering the eye can change by pushing out or pulling in the front face (surface) of the auxiliary member 50 arranged in the peripheral part with the actuator 10. With this, the refractive power can be changed by minus 1 diopter or plus 1 diopter, for example. In this case, the refractive power of plus 1 diopter can be achieved with the change in the radius of curvature by 90.6 mm. This change amount is a very small change of about 0.5 micrometers as the change in the thickness of the central part of the auxiliary member 50 in the light passing direction (about 1 cubic millimeters if converted into volume). As a result, it becomes possible to smoothly perform the adjustment of the ocular refractive power with high precision.

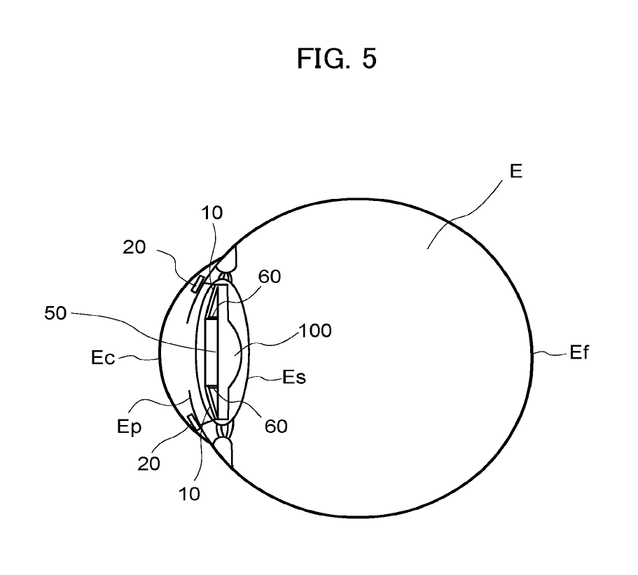

Note that the controller 20 may be arranged on the iris Ep as shown in FIG. 5. The controller 20 can transmit and receive signals to and from the auxiliary member 50 and the intraocular lens 100, which are arranged in the capsular bag, via a wired or wireless signal transmission path. Note that the symbol Es in FIG. 5 shows the capsular bag. In addition, one or more controllers 20 may be placed in the eye, and one or more actuators 10 may be controlled simultaneously or independently. By independently deforming a plurality of actuators 10, it becomes possible to correct the astigmatism or one or more higher order aberrations.

### Operation Examples

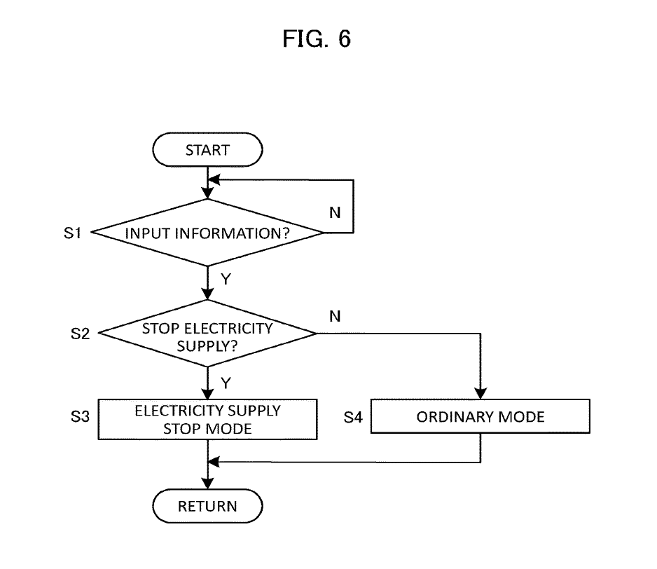

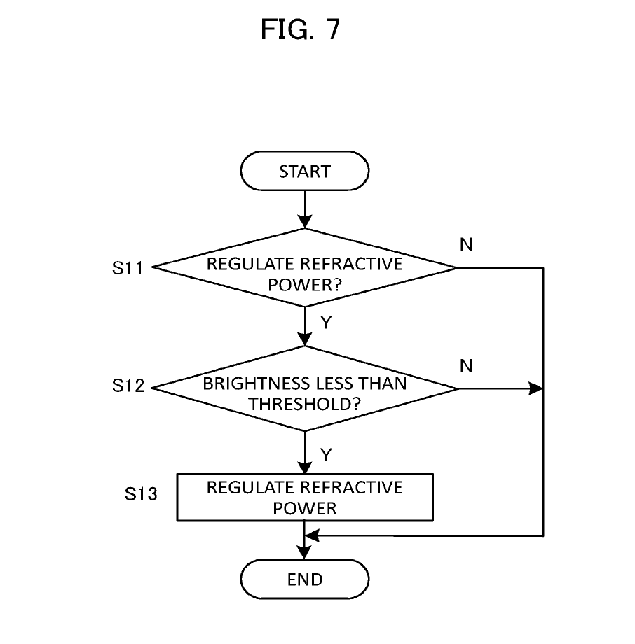

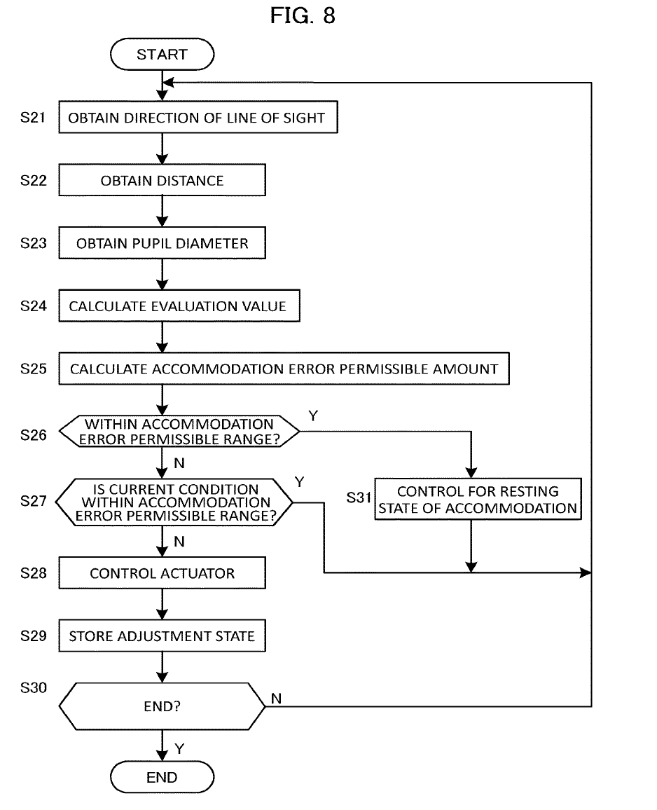

Operation examples of the ocular function assistance device 1 will be described. FIGS. 6, 7, and 8 shows examples of the operation of the ocular function assistance device 1

### (S1)

To begin with, the controller 20 watches and waits the input of the biological information BI or the condition information EI from the information input unit 30 (S1: N). Upon receiving the input of the biological information BI or the condition information EI from the information input unit 30 (S1: Y), the operation of the ocular function assistance device 1 moves on to S2.

### (S2)

Upon receiving the input of the biological information BI or the condition information EI from the information input unit 30 (S1: Y), the controller 20 determines whether or not to stop the electricity supply to the actuator 10 based on the biological information BI or the condition information EI input in S1. For example, the controller 20 stores, in advance, determination information in which one or more information types for stopping the electricity supply are associated with criteria. The controller 20 refers to the determination information to determine whether or not to stop the electricity supply to the actuator 10 based on the biological information BI or the condition information EI. If the electricity supply to the actuator 10 is determined to be stopped (S2: Y), the operation of the ocular function assistance device 1 moves on to S3. If the electricity supply to the actuator 10 is determined not to be stopped (S2: N), the operation of the ocular function assistance device 1 moves on to S4.

### (S3)

If the electricity supply to the actuator 10 is determined to be stopped (S2: Y), the controller 20 switches the operation mode to the electricity supply stop mode. That is, the controller 20 stops the electricity supply to the actuator 10. The operation of the ocular function assistance device 1 moves on to S1 (RETURN).

For example, the controller 20 may be configured to switch the operation mode to the ordinary mode and move the operation of the ocular function assistance device 1 on to S1 if the information input unit 30 inputs predefined second information as the biological information BI or the condition information EI (RETURN). Alternatively, the controller 20 may be configured to switch the operation mode to the ordinary mode and move the operation of the ocular function assistance device 1 on to S1 when a predefined period of time has elapsed (RETURN).

### (S4)

If the electricity supply to the actuator 10 is determined not to be stopped (S2: N), the controller 20 sets the operation mode to the ordinary mode and continues the operation. Examples of the operation of the ordinary mode will be described later. The operation of the ocular function assistance device 1 moves on to S1 (RETURN).

In S4, the ocular function assistance device 1 can execute the following operations.

### (S11)

The controller 20 determines whether or not to adjust the refractive power based on the biological information BI or the condition information EI input by the information input unit 30. For example, the controller 20 stores, in advance, control information in which one or more information types for adjusting the refractive power are associated with criteria. The controller 20 refers to the control information to determine whether or not to adjust the refractive power based on the biological information BI or the condition information EI. If the adjustment of refractive power is determined to be performed (S11: Y), the operation of the ocular function assistance device 1 moves on to S12. If the adjustment of refractive power is determined not to be performed (S11: N), the operation of the ocular function assistance device 1 moves on to S1 in FIG. 1.

### (S12)

If the adjustment of refractive power is determined to be performed (S11: Y), the controller 20 determines whether or not the ambient brightness is less than a predefined threshold based on the condition information EI. The threshold may be set in an arbitrary manner. The threshold may be set based on past control contents (e.g., control history, control record). If the ambient brightness is determined to be less than the threshold (that is, the pupil diameter is assumed to be large) (S12: Y), the operation of the ocular function assistance device 1 moves on to S13. If the ambient brightness is determined to be equal to or larger than the threshold (that is, the pupil diameter is assumed to be small) (S12: N), the operation of the ocular function assistance device 1 moves on to S1 in FIG. 1.

If the ambient brightness is determined to be less than the threshold (S12: Y), the controller 20 determines the adjustment direction and the adjustment amount of the refractive power based on the biological information BI or the condition information EI, and outputs a control signal corresponding to the determined adjustment direction and adjustment amount to the actuator 10. An example of the operation in S13 will be described later. The operation of the ocular function assistance device 1 moves on to S1 in FIG. 1.

In S13, the ocular function assistance device 1 can execute the following operations.

### (S21, S22, S23)

The controller 20 obtains the direction of line of sight, the distance to the object to be watched by the eye, and the pupil diameter from the condition information EI input by the information input unit 30. The direction of line of sight, the distance, the pupil diameter, and the like can be obtained from an image acquired by an imaging device in advance, by the information generation unit 35. It is also possible to determine the state of the pupil diameter based on the ambient brightness. The controller 20 may be configured to determine them from the condition information EI.

### (S24)

Next, the controller 20 determines an evaluation value in which the current distance (e.g., the distance at the time of measurement for creating the condition information EI) is regarded as the accommodation state of minus 1 diopter (i.e., as the resting state of accommodation) based on the direction of line of sight, the distance, the pupil diameter, and the like obtained in S21 to S23. The evaluation value may be modulation transfer function (MTF), Strehl ratio, or the like.

### (S25)

Then, the controller 20 calculates an accommodation error permissible amount according to a predefined algorithm based on the biological information BI or the condition information EI. The accommodation error permissible amount may be defined in advance. The accommodation error permissible amount may be set in an arbitrary manner. The accommodation error permissible amount may be set based on past control contents (e.g., control history, control record).

### (S26)

Next, the controller 20 determines whether or not the evaluation value obtained in S24 is within the accommodation error permissible amount range determined in S25 on the basis of the resting state of accommodation. If the evaluation value is determined to be within the accommodation error permissible amount range (S26: Y), the operation of the ocular function assistance device 1 moves on to S31. If the evaluation value is determined not to be within the accommodation error permissible amount range (S26: N), the operation of the ocular function assistance device 1 moves on to S27.

### (S27)

If the evaluation value is determined not to be within the accommodation error permissible amount range (S26: N), the controller 20 determines whether or not the evaluation value obtained in S24 is within the accommodation error permissible amount range determined in S25 on the basis of the current refractive power adjustment state. If the evaluation value is determined to be within the accommodation error permissible amount range (S27: Y), the operation of the ocular function assistance device 1 moves on to S21 without performing the adjustment of the refractive power. If the evaluation value is determined not to be within the accommodation error permissible amount range (S27: N), the operation of the ocular function assistance device 1 moves on to S28.

### (S28)

If the evaluation value is determined not to be within the accommodation error permissible amount range (S27: N), the controller 20 controls the actuator 10 based on the evaluation value obtained in S24 and the current refractive power adjustment state.

### (S29)

The controller 20 stores the current control contents (current refractive power adjustment state). the operation of the ocular function assistance device 1 moves on to S30. The control contents stored in S29 is used as the current refractive power adjustment state in S27.

### (S30)

When finishing the refractive power regulation (S30: Y), the operation of the ocular function assistance device 1 ends (END). When not finishing the refractive power regulation (S30: N), the operation of the ocular function assistance device 1 moves on to S21.

### (S31)

If the evaluation value is determined to be within the accommodation error permissible amount range (S26: Y), the controller 20 stops the control of the actuator 10 for leading the refractive power to the resting state of accommodation. For example, the controller 20 stops the electricity supply to the actuator 10. The operation of the ocular function assistance device 1 moves on to S21.

As described above, when it is determined to be within the permissible range from the resting state of accommodation, the operation of the actuator 10 is stopped so as to become minus 1 diopter corresponding to the resting state of accommodation of the auxiliary member 50 and the intraocular lens 100. In addition, when it is determined that the refractive power adjustment from the current adjustment state is not necessary, the control of the actuator 10 is not carried out.

### Actions and Effects

The actions and effects of the ocular function assistance device of the present embodiment will be described.

The ocular function assistance device of the embodiment (for example, ocular function assistance device 1) is used for assisting an ocular function. The ocular function assistance device includes an actuator (for example, actuator 10), a controller (for example, controller 20), and an information input unit (for example, information input unit 30). The actuator is used for providing a predefined ocular function by receiving electricity. The controller executes at least control of electricity supply to the actuator. The information input unit input biological information (for example, biological information BI) or condition information (for example, condition information EI) into the controller. The controller changes the control mode of the actuator based on the biological information or the condition information.

With such a configuration, it becomes possible to continue to appropriately maintain the ocular function without imposing a burden on the subject, and provide a novel technique to assist the ocular function.

The controller may be configured to control the electricity supply to the actuator and the electric current direction thereof, and control the electricity supply and the electric current direction based on the biological information or the condition information.

With such a configuration, unnecessary electricity supply to the actuator for providing the ocular function can be stopped according to the electric current direction. Hence, it becomes possible to reduce the electricity consumption of the ocular function assistance device.

The controller may be configured to stop the electricity supply to the actuator when the information input unit inputs first information as the biological information or the condition information.

With such a configuration, unnecessary electricity supply to the actuator is reduced, and electricity consumption for continuing to maintain the ocular function can be reduced.

The actuator may be configured to receive the electricity supply and change the refractive power of the crystalline lens or the intraocular lens from the reference value corresponding to the ocular refractive power of minus 1 diopter.

With such a configuration, for example, the necessity of the operation of the actuator can be eliminated by allowing the state of the refractive power of minus 1 diopter as the resting state of accommodation, and the electricity supply can be stopped during the resting state of accommodation. Hence, it becomes possible to reduce the electricity consumption.

The controller may be configured to set the control content applied at least when the electricity supply has been stopped to be the reference value.

With such a configuration, it becomes possible to learn the states in which the electricity supply is stopped for individual subjects and perform electricity saving according to individual subjects.

The controller may be configured to switch the control mode to the ordinary mode and the electricity supply stop mode.

With such a configuration, it becomes possible to continue to appropriately maintain the ocular function without imposing a burden on the subject by switching the operation mode.

The controller may be configured to switch the control mode to the coarse operation mode for coarsely operating the actuator and the fine operation mode for finely operating the actuator.

With such a configuration, it becomes possible to appropriately assist the ocular function according to the change in the environmental conditions. For example, if it is determined that the pupil diameter is equal to or larger than the threshold based on the condition information, the actuator is controlled to finely adjust the ocular refractive power. On the other hand, if it is determined that the pupil diameter is less than the threshold based on the condition information, the actuator is controlled to coarsely adjust the ocular refractive power.

The actuator may include a shape changing part including a polymeric material whose shape changes upon receiving the electricity, and a pair of electrodes that interposes the shape changing part in between.

With such a configuration, it becomes possible to use an actuator that has a simple structure and is deformable in the direction corresponding to the electric current direction according to the supplied electricity.

The ocular function assistance device of the embodiment may include a power supply unit (for example, power supply unit 40) that receives control from the controller and supplies electricity to the actuator.

With such a configuration, it becomes possible to provide an ocular function assistance device having the power supply unit that supplies electricity to the actuator.

The ocular function assistance device of the embodiment may include an auxiliary member (for example, auxiliary member 50) that can be placed in the eye and provides the predefined ocular function upon receiving the operation of the actuator.

With such a configuration, it becomes possible to provide an ocular function assistance device for assisting the provision of the predefined ocular function with the auxiliary member having received the operation of the actuator.

The actuator and the auxiliary member may be integrally formed.

With such a configuration, it becomes possible to easily implant the actuator and the auxiliary member into the eye.

At least the auxiliary member may have flexibility.

With such a configuration, the auxiliary member is foldable, and hence the implantation into the eye becomes easier.

At least the actuator and the auxiliary member may be placed in the capsular bag.

With such a configuration, it becomes possible to perform the implantation into the capsular bag in the same manner as the standard intraocular lens implantation. In addition, it becomes possible to continue to appropriately maintain the ocular function even when various conditions change in daily life.

At least the actuator and the auxiliary member may be placed between the iris and the crystalline lens.

With such a configuration, it becomes possible to continue to appropriately maintain the ocular function even when various conditions change in daily life, with the crystalline lens remained.

### First Modification Example

In the embodiment described above, the intraocular lens is arranged in the capsular bag. However, the ocular function assistance device according to embodiments are not so limited.

For example, an ocular function assistance device of an embodiment may be inserted as an accommodative intraocular lens of the Implantable Collamer Lens (ICL) type between the iris and the crystalline lens, with the crystalline lens remained as it is. The accommodative intraocular lens is configured to be stretchable with the auxiliary member 50 having almost the same structure as that in the above embodiment, and fixed in the eye with a hook. The hook is configured to be able to transmit motive power and/or signals to the accommodative intraocular lens. The accommodative intraocular lens includes one or more interval fixing parts. The both ends of the interval fixing part support the front face and the rear face of the auxiliary member 50. With this, it is configured that the interval between the front face and the rear face of the auxiliary member 50 becomes a predefined distance or more.

One or more actuators 10 are arranged in the peripheral part on the front face side and the peripheral part on the rear face side of the auxiliary member 50. For example, one end of the actuator 10 arranged in the peripheral part on the front face side is fixed to the edge part of the auxiliary member 50, and the other end is supported by a ring-shaped fixing part provided on the front face side. One end of the actuator 10 arranged in the peripheral part on the rear face side is fixed to the edge part of the auxiliary member 50, and the other end is supported by a ring-shaped fixing part provided on the rear face side.

With such a configuration, the rear face of the auxiliary member 50 can be deformed into a concave shape, and the subtle interval to the front face of the crystalline lens can be maintained and subtle pressing force can be given. As a result, the effect of decreasing the incidence of cataract after the operation can also be expected.

### Second Modification Example

The accommodative intraocular lenses of the ICL type are not limited to those described in the first modification example.

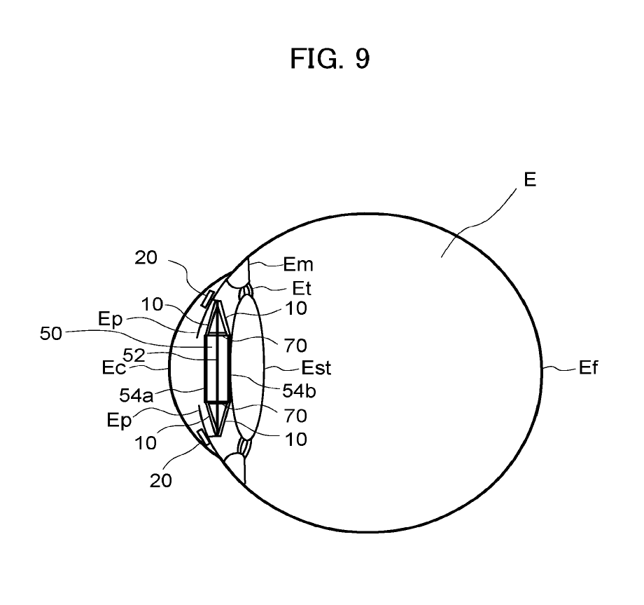

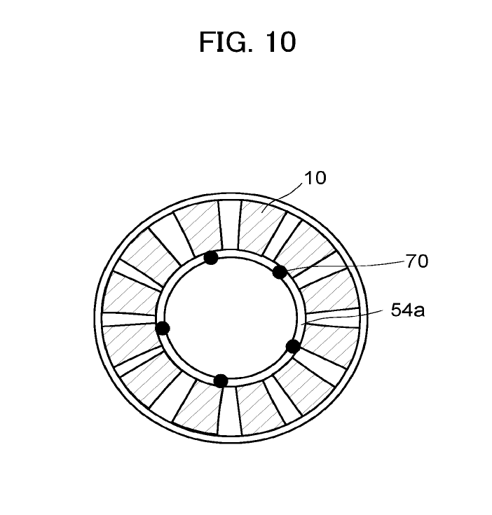

FIG. 9 shows a schematic sectional view of the eye in which the ocular function assistance device according to the second modification example of the embodiment is implanted. FIG. 10 shows a schematic diagram of an example of the arrangement of the actuator according to the second modification example of the embodiment. FIG. 10 is a view of the actuator 10 of the embodiment as seen from the front side of the eye. In FIG. 9, the symbol Est shows the crystalline lens, the symbol Em shows the ciliary body, and the symbol Et shows the Zinn's zonule. In FIG. 9, the same parts as in FIG. 2 are shown by the same symbols and their descriptions will be omitted in an appropriate manner. In FIG. 10, the same parts as in FIG. 9 are shown by the same symbols and their descriptions will be omitted in an appropriate manner.

Like the first modification example, the accommodative intraocular lens 110 according to the second modification example is inserted between the iris and the crystalline lens with the crystalline lens remained as it is. The accommodative intraocular lens 110 is configured to be stretchable with the auxiliary member 50 having almost the same configuration as that in the above embodiment, and the peripheral part of the auxiliary member 50 is held by the shape maintaining part 51. The accommodative intraocular lens 110 includes one or more interval fixing parts 70. The both ends of the interval fixing part 70 support the front face and the rear face of the auxiliary member 50. With this, it is configured that the interval between the front face and the rear face of the auxiliary member 50 becomes a predefined distance or more.

One or more actuators 10 are arranged in the peripheral part on the front face side and the peripheral part on the rear face side of the auxiliary member 50. For example, one end of the actuator 10 arranged in the peripheral part on the front face side is fixed to the edge part of the auxiliary member 50, and the other end is supported by the ring-shaped fixing part 54a provided on the front face side. One end of the actuator 10 arranged in the peripheral part on the rear face side is fixed to the edge part of the auxiliary member 50, and the other end is supported by the ring-shaped fixing part 54b provided on the rear face side.

The embodiments and modification examples described above are merely examples for implementing the present invention. Therefore, it is possible to make arbitrary modifications within the scope of the present invention.

It is desired that the members according to the embodiments or modification examples described above are transparent members that do not impede the transmission of the light entering the eye.

The actuator in the embodiments or modification examples described above may be formed in a ring shape and have one or more rifts to be bendable.

### EXPLANATION OF SYMBOLS

1 ocular function assistance device
10 actuator
20 controller
30 information input unit
35 information generation unit

1. An ocular function assistance device for assisting an ocular function, comprising:
an actuator configured to receive electricity and operate for providing a predefined ocular function;
a controller configured to execute at least control of electricity supply to the actuator; and
an information input unit configured to input biological information or condition information into the controller,
wherein the controller changes a control mode of the actuator based on the biological information or the condition information.

2. The ocular function assistance device of claim 1, wherein the controller controls the electricity supply to the actuator and an electric current direction thereof, and controls the electricity supply and the electric current direction based on the biological information or the condition information.

3. The ocular function assistance device of claim 1, wherein the controller stops the electricity supply to the actuator when the information input unit inputs first information as the biological information or the condition information.

4. The ocular function assistance device of claim 1, wherein the actuator receives the electricity supply and changes refractive power of a crystalline lens or an intraocular lens from a reference value corresponding to ocular refractive power of minus 1 diopter.

5. The ocular function assistance device of claim 4, wherein the controller sets a control content applied at least when the electricity supply has been stopped as the reference value.

6. The ocular function assistance device of claim 1, wherein the controller can switch the control mode to an ordinary mode and an electricity supply stop mode.

7. The ocular function assistance device of claim 1, wherein the controller can switch the control mode to a coarse operation mode for coarsely operating the actuator and a fine operation mode for finely operating the actuator.

8. The ocular function assistance device of claim 1, wherein the actuator comprises:
a shape changing part comprising a polymeric material whose shape changes upon receiving the electricity; and
a pair of electrodes that interposes the shape changing part in between.

9. The ocular function assistance device of claim 1, further comprising a power supply unit configured to receive control from the controller and supply electricity to the actuator.

10. The ocular function assistance device of claim 1, further comprising an auxiliary member configured to be capable of being placed in an eye, and provide the predefined ocular function upon receiving an operation of the actuator.

11. The ocular function assistance device of claim 10, wherein the actuator and the auxiliary member are integrally formed.

12. The ocular function assistance device of claim 10, wherein at least the auxiliary member has flexibility.

13. The ocular function assistance device of claim 10, wherein at least the actuator and the auxiliary member are placed in a capsular bag.

14. The ocular function assistance device of claim 10, wherein at least the actuator and the auxiliary member are placed between an iris and a crystalline lens.
